(19) United States
(12) Patent Application Publication
(10) Pub. No.: US 2024/0285530 A1
(43) Pub. Date: Aug. 29, 2024

(54) REDUCING HYDROPHOBIC DRUG ADSORPTION IN ECMO CIRCUITS

(71) Applicant: UNIVERSITY OF UTAH RESEARCH FOUNDATION, Salt Lake City, UT (US)

(72) Inventors: Nitish KHURANA, Salt Lake City, UT (US); Hamidreza GHANDEHARI, Salt Lake City, UT (US); Kevin WATT, Salt Lake City, UT (US); Venkata YELLEPEDDI, Salt Lake City, UT (US)

(21) Appl. No.: 18/112,720

(22) Filed: Feb. 22, 2023

(51) Int. Cl. A61K 9/107; A61K 9/127; A61K 31/05

(52) U.S. Cl. CPC A61K 9/1075; A61K 9/1271; A61K 31/05; B82Y 5/00; B82Y 30/00; B82Y 40/00

(57) ABSTRACT

Described herein are methods for administering a hydrophobic pharmaceutical agent to subjects undergoing extracorporeal membrane oxygenation (ECMO) device therapy while diminishing adsorption of the hydrophobic pharmaceutical agent to the ECMO device. In one embodiment, the method comprises administering the hydrophobic pharmaceutical agent encapsulated in an amphipathic encapsulating agent. In one aspect, the hydrophobic pharmaceutical agent is propofol and the amphipathic encapsulating agent is polyethylene-polypropylene glycol (Poloxamer 407) or a PEGylated liposome. In another aspect, PEGylated propofol is synthesized and used to prevent adsorption to the ECMO device.

### CROSS REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### REFERENCE TO SEQUENCE LISTING

Not applicable.

### TECHNICAL FIELD

Described herein are methods for administering a hydrophobic pharmaceutical agent to subjects undergoing extracorporeal membrane oxygenation (ECMO) device therapy while diminishing adsorption of the hydrophobic pharmaceutical agent to the ECMO device. In one embodiment, the method comprises administering the hydrophobic pharmaceutical agent encapsulated in an amphipathic encapsulating agent. In one aspect, the hydrophobic pharmaceutical agent is propofol and the amphipathic encapsulating agent is polyethylene-polypropylene glycol (Poloxamer 407) or a PEGylated liposome. In another aspect, PEGylated propofol is synthesized and used to prevent adsorption to the ECMO device.

### BACKGROUND

Extracorporeal membrane oxygenation (ECMO) is a life-saving cardiopulmonary bypass device used in critically ill patients with refractory heart and lung failure. Patients supported with ECMO receive numerous drugs to treat critical illness and the underlying disease. Unfortunately, the majority of drugs prescribed to patients on ECMO lack dosing information. Preliminary data demonstrates that dosing is different in this population because the ECMO circuit components can adsorb drugs and affect drug exposure substantially. Propofol is a widely used anesthetic in ECMO patients and is known to have high adsorption rates in ECMO circuits due to its high hydrophobicity.

What is needed are compositions and methods for preventing hydrophobic drugs such as propofol from adsorbing to ECMO devices and reducing the dose administered.

### SUMMARY

One embodiment described herein is a pharmaceutical composition for administration in extracorporeal membrane oxygenation (ECMO) circuits, the composition comprising propofol encapsulated in an amphipathic encapsulating agent. In one aspect, the amphipathic encapsulating agent comprises a polymeric micelle or a liposome. In another aspect, the polymeric micelle comprises a polyethylene-polypropylene glycol micelle. In another aspect, the polyethylene-polypropylene glycol micelle is Poloxamer 407 (P407). In another aspect, the polyethylene-polypropylene glycol micelle is Poloxamer 188 (P188). In another aspect, the polyethylene-polypropylene glycol micelle is a mixture of P407 and P188. In another aspect, the liposome comprises a PEGylated liposome. In another aspect, the composition is stable for at least about 7 days at about 37° C. In another aspect, the composition is stable for at least about 6 months at about 2° C. to about 8° C. In another aspect, the composition has reduced adsorption in ECMO circuits as compared to compositions comprising propofol that is not encapsulated in an amphipathic encapsulating agent. In another aspect, the polymeric micelle encapsulating the propofol has a size of about 20 nm to about 40 nm. In another aspect, the polymeric micelle encapsulating the propofol has a size of about 25 nm to about 35 nm. In another aspect, the polymeric micelle encapsulating the propofol has a size of about 20 nm to about 30 nm. In another aspect, the polymeric micelle encapsulating the propofol has a size of about 30 nm to about 40 nm. In another aspect, the polymeric micelle encapsulating the propofol has a polydispersity index of about 0.05 to about 0.5. In another aspect, the polymeric micelle encapsulating the propofol has a polydispersity index of about 0.05 to about 0.2. In another aspect, the polymeric micelle encapsulating the propofol has a zeta potential of about-2.50 mV to about-5.00 mV. In another aspect, the polymeric micelle is present in an amount of about 5 wt % to about 14 wt %. In another aspect, the liposome encapsulating the propofol has a size of about 120 nm to about 400 nm. In another aspect, the liposome encapsulating the propofol has a polydispersity index of about 0.05 to about 0.5. In another aspect, the propofol is present in an amount of about 5 mg/mL to about 15 mg/mL. In another aspect, the propofol is present in an amount of about 10 mg/mL.

Another embodiment described herein is a method for encapsulating propofol in an amphipathic encapsulating agent, the method comprising: (a) combining propofol and the amphipathic encapsulating agent to create a mixture; and (b) agitating the mixture for a period of time sufficient to encapsulate the propofol in the amphipathic encapsulating agent. In one aspect, the amphipathic encapsulating agent comprises a polymeric micelle or a liposome. In another aspect, the polymeric micelle is present in an amount of about 5 wt % to about 14 wt %. In another aspect, the propofol is present in an amount of about 5 mg/ml to about 15 mg/mL. In another aspect, agitating the mixture is performed at about 4° C. In another aspect, agitating the mixture comprises sonication. In another aspect, the period of time is about 30 minutes to about 16 hours. In another aspect, the method further comprises filtering the amphipathic encapsulating agent encapsulating the propofol. In another aspect, the method has an encapsulation efficiency of at least about 90%. In another aspect, the method has an encapsulation efficiency of about 94% to about 98%.

Another embodiment described herein is a method for administering a hydrophobic pharmaceutical agent to a subject undergoing extracorporeal membrane oxygenation (ECMO) device therapy while diminishing adsorption of the hydrophobic pharmaceutical agent to the ECMO device, the method comprising administering the hydrophobic pharmaceutical agent encapsulated in an amphipathic encapsulating agent. In one aspect, the amphipathic encapsulating agent comprises a polymeric micelle or a liposome. In another aspect, the polymeric micelle comprises a polyethylene-polypropylene glycol micelle selected from Poloxamer 407 (P407), Poloxamer 188 (P188), or a combination thereof. In another aspect, the hydrophobic pharmaceutical agent is propofol. In another aspect, the hydrophobic pharmaceutical agent encapsulated in the amphipathic encapsulating agent is stable for at least about 7 days at about 37° C. In another aspect, the hydrophobic pharmaceutical agent encapsulated in the amphipathic encapsulating agent is administered to the subject at a dose of about 0.5 mg/kg to about 5 mg/kg. In another aspect, the hydrophobic pharmaceutical agent encapsulated in the amphipathic encapsulating agent is administered to the subject over a period of about 1 minute to about 7 days.

Another embodiment described herein is a composition of PEGylated propofol having the structure of Formula I:

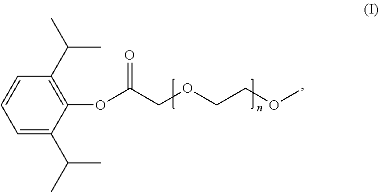

where n is 40-50. In one aspect, n is 45 (PEG mol wt of ˜2000 Da).

Another embodiment described herein is a method for synthesizing a PEGylated propofol as described herein, the method comprising: combining propofol, polyethyleneglycol acetic acid (MW: 2000 Da), 4-dimethylaminopyridine (DMAP), and diisopropylcarbodiimide (DIC) in dichloromethane (DCM); permitting the reaction to proceed for 18-24 hours; and extracting the PEGylated propofol using aqueous and organic extractions.

Another embodiment described herein is a method for administering a propofol to a subject undergoing extracorporeal membrane oxygenation (ECMO) device therapy while diminishing adsorption of the propofol to the ECMO device, the method comprising administering PEGylated propofol. In one aspect, the PEGylated propofol is administered to the subject at a dose of about 0.5 mg/kg to about 5 mg/kg. In another aspect, the PEGylated propofol is administered to the subject over a period of about 1 minute to about 7 days.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show the polydispersity Index (PDI) (FIG. 1A) and size (FIG. 1B) of propofol loaded and free micelles. Data represent the mean (n=3)±SD. **P<0.01.

FIG. 2 shows a Scanning Electron Microscopy (SEM) image of micelles.

FIG. 3 shows the stability of propofol loaded micelles. Size and PDI of micelles were characterized for 7 days. Data represent the mean (n=3)±SD.

FIG. 4 shows a calibration curve of propofol in serum.

FIG. 5A-B shows the viability of THP-1 cells (FIG. 5A) and RAW 264.7 cells (FIG. 5B) at varying concentrations of propofol (n=6). Data represents the mean±SD. *P<0.05; **P<0.01; ***P<0.001; ****P<0.0001.

FIG. 6 shows the recovery of propofol from micellar propofol and Diprivan® at different time points. A bolus injection was given at T=0 and Infusion dose was started at T=4 hr and stopped at T=6 hr with samples collected for a total of 10 hrs. Data represent the mean (n=3)+SD. Data and statistics are shown in Table 3, below.

FIG. 7 shows a study design for evaluation of propofol adsorption in an ex vivo ECMO circuit setup.

FIG. 8A-B show tilt tests that were performed to evaluate the gelation kinetics for different P407 formulations at different temperatures. FIG. 8A shows tilt test results for formulations having concentrations of P407 at 10%, 11%, 12%, 13%, 14%, 15%, 16%, 17%, 18%, and 19% P407 performed at 4° C., 25° C., and 37° C. for 15 sec, 30 sec, 1 min, and 4 min. FIG. 8B shows tilt test results for a formulation having 16% P407 performed at 37° C. for 10 min, 15 min, 30 min, 60 min, and 120 min.

FIG. 9 shows a calibration curve of meropenem determined at a wavelength of 250 nm.

FIG. 10A-C show infrared spectroscopy performed using a Bruker Alpha attenuated total reflectance Fourier transformed infrared (ATR-FTIR). FIG. 10A shows the FTIR-OH (broad) spectrum for propofol, PEG, and Rxn4 (propofol-PEG). FIG. 10B shows the FTIR-C-O-C-stretch spectrum for propofol, PEG, and Rxn4. FIG. 10C shows the FTIR-1,2,3-trisubstituted aryl spectrum for propofol, PEG, and Rxn4.

FIG. 11A-C show ¹H nuclear magnetic resonance (NMR) spectroscopy performed using a Varian mercury spectrophotometer (400 Hz). The chemical shifts were reported relative to deuterated solvent (CDCl₃). FIG. 11A shows the NMR spectrum for propofol. FIG. 11B shows the NMR spectrum for methoxy-PEG acetic acid. FIG. 11C shows the NMR spectrum for propofol-PEG.

FIG. 12 shows UV-vis spectroscopy of PEG, propofol, and PEG-propofol performed using a Spectramax M2 microplate reader (Molecular Devices).

### DETAILED DESCRIPTION

Unless otherwise defined, all technical and scientific terms used herein have the same meaning as commonly understood by one of ordinary skill in the art. For example, any nomenclatures used in connection with, and techniques of medicine, biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “amphipathic encapsulating agent” refers to a molecule having both hydrophilic (i.e., polar) and hydrophobic (i.e., nonpolar) lipid components that may be used to encapsulate one or more drugs. In some embodiments of the present invention, the amphipathic encapsulating agent may comprise a polymeric micelle or a liposome. Liposomes are composed of a bilayer of amphipathic molecules where the two layers of molecules are arranged in two concentric circles. Micelles are closed lipid monolayers where fatty acids are either present in the core or at the surface. In some embodiments of the present invention, the polymeric micelle may comprise a polyethylene-polypropylene glycol micelle selected from Poloxamer 407 (P407), Poloxamer 188 (P188), or a mixture or combination of P407 and P188. In some embodiments of the present invention, the liposome may comprise a PEGylated liposome generated from 1,2-dipalmitoyl-sn-glycero-3-phosphocholine (DPPC), cholesterol, and 1,2-distearoyl-snglycero-3-phosphoethanolamine-N-[methoxy(poly (ethylene glycol))-2000] (DSPE-PEG2000).

Described herein are compositions and methods for preventing the adsorption of hydrophobic drugs to extracorporeal membrane oxygenation (ECMO) devices by encapsulating the drug in a polymer micelle. The hydrophobicity of the drug must be matched with the polymers used to make the micelle. This is empirical and must be optimized for each drug or classes of drugs with similar hydrophobicity and classes of polymers.

One embodiment described herein is a method for administering a hydrophobic pharmaceutical agent to subjects undergoing ECMO device therapy while diminishing adsorption of the hydrophobic pharmaceutical agent to the ECMO device, the method comprising administering the hydrophobic pharmaceutical agent encapsulated in polymeric micelles. In one aspect, the hydrophobic pharmaceutical agent is propofol and the polymeric micelles are prepared from polyethylene-polypropylene glycol (Poloxamer 407). In one aspect, the method comprises administering propofol to subjects undergoing ECMO device therapy while diminishing adsorption of the propofol to the ECMO device, the method comprising administering propofol encapsulated in polyethylene-polypropylene glycol micelles (e.g., Poloxamer 407).

Propofol was encapsulated within polyethylene-polypropylene glycol (e.g., Poloxamer 407, Pluronic® F127 (BASF), Kolliphor® P407 (BASF Pharma). An encapsulation efficiency of 96.2±1.3% was obtained. Size characterization was performed using dynamic light scattering and the encapsulated propofol micelles had sizes of 28 nm with 0.08 polydispersity. The micelles were stable at physiological temperature (37° C.) for a period of at least 7-days. These micelles were compared to a propofol emulsion (Diprivan®) in an ex-vivo ECMO system. Micellar propofol demonstrated a significant reduction in adsorption of propofol in the ECMO circuit at earlier time points compared to Diprivan®.

Another embodiment described herein is a composition comprising propofol encapsulated in polyethylene-polypropylene glycol micelles (Poloxamer 407).

Another embodiment described herein is a method for manufacturing a composition comprising propofol encapsulated in polyethylene-polypropylene glycol micelles (Poloxamer 407) and the composition prepared by the method.

One embodiment described herein is a pharmaceutical composition for administration in extracorporeal membrane oxygenation (ECMO) circuits, the composition comprising propofol encapsulated in an amphipathic encapsulating agent. In one aspect, the amphipathic encapsulating agent comprises a polymeric micelle or a liposome. In another aspect, the polymeric micelle comprises a polyethylene-polypropylene glycol micelle. In another aspect, the polyethylene-polypropylene glycol micelle is Poloxamer 407 (P407). In another aspect, the polyethylene-polypropylene glycol micelle is Poloxamer 188 (P188). In another aspect, the polyethylene-polypropylene glycol micelle is a mixture of P407 and P188. In another aspect, the liposome comprises a PEGylated liposome. In another aspect, the composition is stable for at least about 7 days at about 37° C. In another aspect, the composition is stable for at least about 6 months at about 2° C. to about 8° C. In another aspect, the composition has reduced adsorption in ECMO circuits as compared to compositions comprising propofol that is not encapsulated in an amphipathic encapsulating agent. In another aspect, the polymeric micelle encapsulating the propofol has a size of about 20 nm to about 40 nm. In another aspect, the polymeric micelle encapsulating the propofol has a size of about 25 nm to about 35 nm. In another aspect, the polymeric micelle encapsulating the propofol has a size of about 20 nm to about 30 nm. In another aspect, the polymeric micelle encapsulating the propofol has a size of about 30 nm to about 40 nm. In another aspect, the polymeric micelle encapsulating the propofol has a polydispersity index of about 0.05 to about 0.5. In another aspect, the polymeric micelle encapsulating the propofol has a polydispersity index of about 0.05 to about 0.2. In another aspect, the polymeric micelle encapsulating the propofol has a zeta potential of about −2.50 mV to about −5.00 mV. In another aspect, the polymeric micelle is present in an amount of about 5 wt % to about 14 wt %. In another aspect, the polymeric micelle is present in an amount of about 6 wt %. In another aspect, the liposome encapsulating the propofol has a size of about 120 nm to about 400 nm. In another aspect, the liposome encapsulating the propofol has a polydispersity index of about 0.05 to about 0.5. In another aspect, the propofol is present in an amount of about 5 mg/mL to about 15 mg/mL. In another aspect, the propofol is present in an amount of about 10 mg/mL.

Another embodiment described herein is a method for encapsulating propofol in an amphipathic encapsulating agent, the method comprising: (a) combining propofol and the amphipathic encapsulating agent to create a mixture; and (b) agitating the mixture for a period of time sufficient to encapsulate the propofol in the amphipathic encapsulating agent. In one aspect, the amphipathic encapsulating agent comprises a polymeric micelle or a liposome. In another aspect, the polymeric micelle is present in an amount of about 5 wt % to about 14 wt %. In another aspect, the propofol is present in an amount of about 5 mg/ml to about 15 mg/mL. In another aspect, agitating the mixture is performed at about 4° C. In another aspect, agitating the mixture comprises sonication. In another aspect, the period of time is about 30 minutes to about 16 hours. In another aspect, the method further comprises filtering the amphipathic encapsulating agent encapsulating the propofol. In another aspect, the method has an encapsulation efficiency of at least about 90%. In another aspect, the method has an encapsulation efficiency of about 94% to about 98%.

Another embodiment described herein is a method for administering a hydrophobic pharmaceutical agent to a subject undergoing extracorporeal membrane oxygenation (ECMO) device therapy while diminishing adsorption of the hydrophobic pharmaceutical agent to the ECMO device, the method comprising administering the hydrophobic pharmaceutical agent encapsulated in an amphipathic encapsulating agent. In one aspect, the amphipathic encapsulating agent comprises a polymeric micelle or a liposome. In another aspect, the polymeric micelle comprises a polyethylene-polypropylene glycol micelle selected from Poloxamer 407 (P407), Poloxamer 188 (P188), or a combination thereof. In another aspect, the hydrophobic pharmaceutical agent is propofol. In another aspect, the hydrophobic pharmaceutical agent encapsulated in the amphipathic encapsulating agent is stable for at least about 7 days at about 37° C. In another aspect, the hydrophobic pharmaceutical agent encapsulated in the amphipathic encapsulating agent is administered to the subject at a dose of about 0.5 mg/kg to about 5 mg/kg. In another aspect, the hydrophobic pharmaceutical agent encapsulated in the amphipathic encapsulating agent is administered to the subject over a period of about 1 minute to about 7 days.

Another embodiment described herein is a composition of PEGylated propofol having the structure of Formula I:

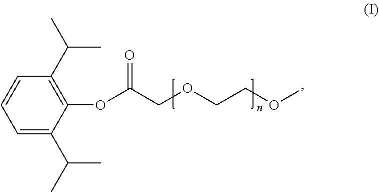

where n is 40-50. In one aspect, n is 45 (PEG mol wt of ˜2000 Da).

Another embodiment described herein is a method for synthesizing a PEGylated propofol as described herein, the method comprising: combining propofol, polyethyleneglycol acetic acid (MW: 2000 Da), 4-dimethylaminopyridine (DMAP), and diisopropylcarbodiimide (DIC) in dichloromethane (DCM); permitting the reaction to proceed for 18-24 hours; and extracting the PEGylated propofol using aqueous and organic extractions.

Another embodiment described herein is a method for administering a propofol to a subject undergoing extracorporeal membrane oxygenation (ECMO) device therapy while diminishing adsorption of the propofol to the ECMO device, the method comprising administering PEGylated propofol. In one aspect, the PEGylated propofol is administered to the subject at a dose of about 0.5 mg/kg to about 5 mg/kg. In another aspect, the PEGylated propofol is administered to the subject over a period of about 1 minute to about 7 days.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

### Formulation and Characterization of Empty Micelles and Propofol Loaded Micelles

To prepare blank micelles, 600 mg of Poloxamer 407 (Kolliphor® P407, BASF, Germany) was added to 10 mL PBS (pH 7.4, VWR Life Science, USA) solution in a glass vial to obtain a 6% (w/v) formulation of P407 micelles. For propofol loaded micelles, propofol (2,6-Diidopropylphenol, Sigma Aldrich, USA) was then added to the glass vials to obtain a desired concentration of 10 mg/mL of propofol. The mixture was placed under constant rotation overnight at 100 RPM in 4° C. using a magnetic stirrer (VWR, USA). The micellar solution was then filtered through 0.22 μm hydrophobic PTFE membrane filters (Membrane solutions, USA). Mean hydrodynamic diameter, polydispersity index (PDI) and zeta-potential were measured by Dynamic Light Scattering (DLS) using a Malvern Zetasizer Nano-ZS (Malvern, Worcestershire, UK).

### Scanning Electron Microscopy

Secondary and backscattered electron images were collected using a Teneo SEM system (Thermo Fisher USA). SEM beam energy and current were optimized for high resolution imaging. Samples were mounted on an aluminum stub using a double-sided carbon tape.

### Determination of Encapsulation Efficiency

The amount of propofol loaded in the micelles was determined using High Performance Liquid Chromatography (HPLC). For HPLC, HP Agilent 1100 instrument was used with a C18 column (Alltima, Hichrome, UK). The injection volume was set to 20 μL with a flow rate of 0.7 mL/min. The wavelength used for UV detection was 270 nm. The mobile phase consisted of 70% (v/v) acetonitrile, 20% (v/v) methanol and 10% (v/v) deionized water. A calibration curve was generated as a function of different propofol concentrations. Propofol-loaded micelles were passed through 0.22 μm hydrophobic PTFE membrane filters (Membrane solutions, USA) twice to remove any unencapsulated propofol from the solution. The solution was then incubated in a tube with hydrophobic surface to enable unencapsulated propofol to adsorb. Encapsulation efficiency (EE %) of propofol was determined using HPLC and calculated by the following equation:

$$\% Encapsulation efficiency = \frac{A mount of drug in micelles ( mg )}{Initital amount of drug ( mg )} \times 100 .$$

### Micellar and Drug Stability

For colloidal stability, the micellar solution in PBS was stored under constant shaking at 100 rpm and at 37° C. for one week in an incubator shaker (Amerex Instruments, USA). Aliquots were collected directly after manufacturing and at each of the following 7 days, and subsequently characterized for their size and PDI via DLS. For chemical stability of the drug, aliquots of the micellar propofol solution on Day 0 and Day 7 were collected. They were then analyzed by HPLC to quantify the amount of drug. Long term storage stability was also measured for the micellar formulations. The micellar solution in PBS was stored for 6 months at 4° C. DLS readings were taken at day 0, 30, 90, and 180.

### In Vitro Cytocompatibility

RAW cells were cultured in RPMI 1640 media supplemented with 10% Fetal Bovine Serum (FBS) with a 5% CO₂ atmosphere at 37° C. 10,000 cells per well were seeded in a 96-well plate for the viability assay for 24 hrs. THP-1 cells were cultured in RPMI 1640 media supplemented with 10% FBS and 0.05 mM 2-Mercapoethanol with a 5% CO₂ atmosphere at 37° C. For differentiation of cells, RPMI media (with FBS) was supplemented with Phorbol 12-myrisate 13-acetate (PMA) at 50 ng/ml. 40,000 cells per well were seeded in a 96-well plate for the viability assay for 24 hr. On the next day, the media containing PMA was removed and fresh media was added for the resting phase of THP-1 cells before exposure to different formulations. After 24 hrs, cells were exposed to different formulations of propofol (micellar propofol, Diprivan®, and free propofol) at different concentrations (30-170 UM) and incubated for 24 hrs. The media was then replaced by media containing 10% (v/v) CCK-8 reagent (DoJindo Molecular Technologies, USA). The cells were incubated for an hour and absorbance was measured at 450 nm using the Spectramax M2 plate reader (Molecular Devices, USA). Cell treated with only media and no propofol formulations were considered as negative control and cells treated with 5% (v/v) DMSO were considered as positive controls. Each experiment was performed in replicates of 6 (n=6).

Ex vivo ECMO Adsorption Evaluation

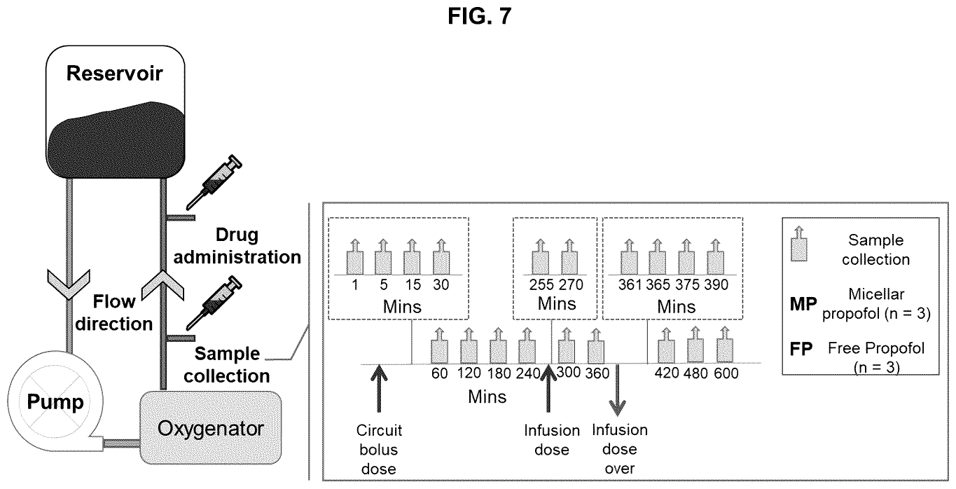

ECMO circuits were assembled according to standard practice with a Quadrox iD oxygenator with bioline coating (Getinge, Sweden), Rotaflow R32 pump with bioline coating (Getinge, Sweden), and custom perfusion tubing with Cortiva™ BioActive Surface (Medtronic, Ireland). Circuits were set up in a closed loop with an IV bag representing the “subject.” Circuits were primed with human packed red blood cells (330 mL), fresh frozen plasma (150 mL), plasmalyte (100 mL, Baxter, USA), heparin (250 units), sodium bicarbonate (3.5 mEq, Civica Rx, USA), tromethamine (2 g, Sigma Aldrich, USA), calcium gluconate (325 mg, Fresenius Kabi, Germany) and albumin (6.25 g, Baxter, USA) and adjusted to maintain physiological conditions. Flow rate was set to 1 L/min to mimic flow for a 10 kg child. For the control, 30 mL of primed blood was drawn from the ECMO circuit and transferred to a falcon tube and maintained at 37° C. in a water bath. Both the ECMO circuit and control were dosed with free propofol and micellar propofol at time 0 to achieve a target concentration of 50 μg/mL (for clinical relevance). Samples were collected at different time points (1, 5, 15, 30, 60, 120, 180 and 240 mins). After the 240-minute sample, the ECMO circuit was dosed with a continuous infusion of 6 mg/hr representing the low end of dosing for a 10 kg child. The infusion was discontinued after 2 hours. Samples were collected at the following time points: 15, 30, 60, 120, 121, 125, 135, 150, 180, 240 and 360 mins after the start of the infusion. Collected blood samples were centrifuged immediately at 3000×g, 4° C.) and plasma samples were stored at −80° C. until ready for further analysis. The samples were then analyzed for propofol concentration using HPLC, and then converted to propofol remaining (propofol recovery) using the following equation:

$$Recovery ( \% ) = \frac{{C}_{t}}{{C}_{i}} \times 100 ,$$

where Cₜ is concentration at time t and Cᵢ is concentration at time=1 min for the bolus dose and Cᵢ is concentration at time=15 mins for the infusion dose. All circuits and controls were run in triplicate. A schematic for ex vivo ECMO setup and sample collection is shown in FIG. 7.

### Statistics

GraphPad Prism 8.0 (GraphPad Software LLC, USA) was used to run statistics. Replicates for each experiment are defined in their respective sections. Unpaired 2-tailed student t-test was employed to run statistics between 2 groups and the threshold of significance was set at P<0.05 (at the 95% Confidence Interval).

### Example 2

### Micelle Formulation and Characterization

For micellar formulation, polyethylene-polypropylene glycol (e.g., Poloxamer 407 or Pluronic® F127) was used. Multiple micellar formulation methods were investigated. The constant agitation method was used owing to its consistent reproducibility. Different concentrations of Poloxamer 407 were tested to optimize the micellar stability, monodisperse size distribution, and encapsulation efficiency. A concentration of 6% (w/v) Poloxamer 407 gave the most consistent results in terms of parameters that were mentioned previously.

Propofol was incorporated into the micelles by adding a 10 mg/mL solution of propofol while mixing 6% (w/v) Poloxamer 407 with phosphate buffered saline (PBS; 137 mM NaCl, 2.7 mM KCl, 4.3 mM Na₂HPO₄, 1.47 mM KH₂PO₄, PH 7.4). A concentration of 10 mg/mL propofol was selected because the clinical formulation of propofol, Diprivan®, is available at same concentration. Micelles were obtained after the mixture was kept at 4° C. overnight with constant agitation a 100 RPM.

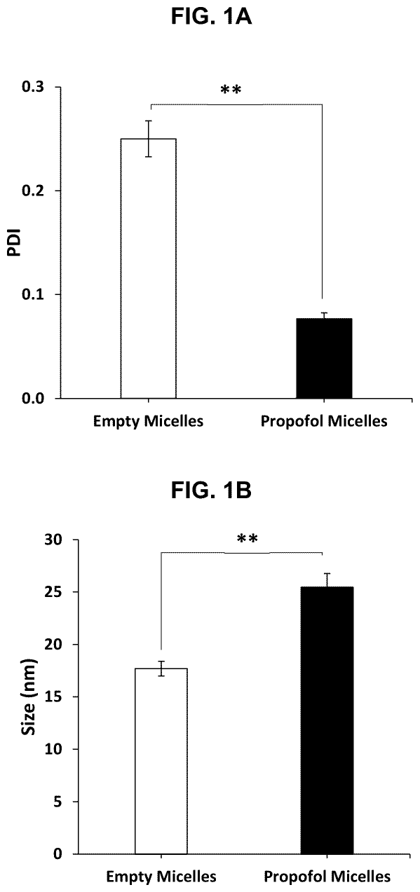

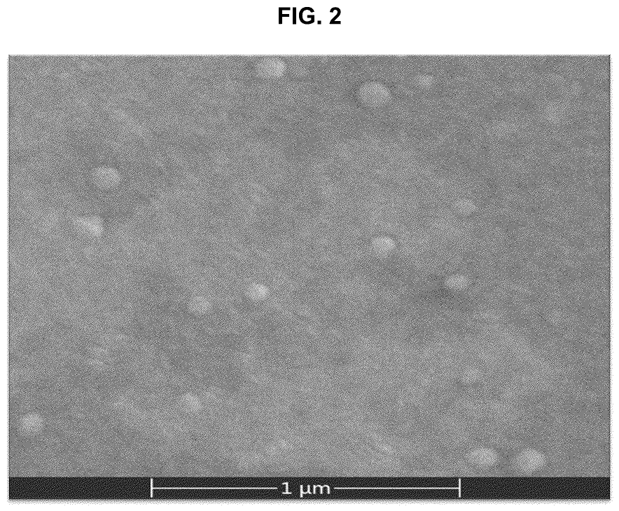

The size and polydispersity index (PDI) were evaluated for all batches of micelles using Dynamic Light Scattering (DLS). FIG. 1 illustrates the size and zeta potential of propofol loaded and free micelles. The zeta potential was also investigated (Table 1). Micelles incorporating propofol were also characterized for their morphology using SEM imaging. As can be observed in FIG. 2, discrete micellar structures with spherical shape were obtained.

TABLE 1
Zeta Potential of Micelles

| Sample | Zeta potential | Standard deviation |
|---|---|---|
| 2 (1:1-1 mM NaCl) | −3.70 mV | 1.00 mV |

### Stability-Physiological Temperature

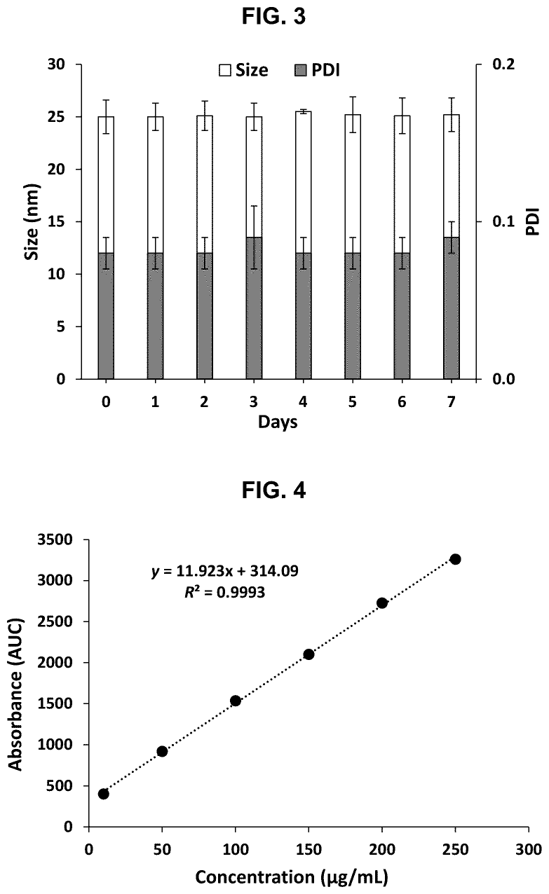

Propofol-loaded micelles were incubated at 37° C. with constant agitation at 100 RPM for 7 days. An aliquot was collected every day up to 7 days and was characterized using DLS for size and PDI. No change in size and PDI were observed and indicates that the micelles are stable physiological temperatures for at least a week (FIG. 3).

### Lyophilization

For the long-term stability of micelles, lyophilization was investigated for the propofol-loaded micelles. Lyophilization helps in making a powder form of the formulation, hence increasing the shelf-life. To protect the formation of ice crystals in micelles, cryoprotectants were added to the formulation during lyophilization. Different cryoprotectants were investigated, however, none of them gave desired results, as the size and PDI of micelles after lyophilization increased significantly. Therefore, the propofol micelles were stored in PBS at 2-8° C. for long-term storage.

### Stability and Long-Term Storage

Propofol-loaded micelles were kept at 2-8° C. in PBS for a period of 6 months. An aliquot was collected at different time points and characterized for size and polydispersity using DLS. There was no change in size and PDI indicating stability of micelles in storage at 2-8° C. (Table 2). This is an ongoing study, and aliquots will be collected at the 1-year time point.

TABLE 2
Size and PDI of Micelles Over Time. Data represent
the mean (n = 3) ± SD

| Time (months) | P407 | Propofol | Z-Ave (d, nm) | PDI |
|---|---|---|---|---|
| 0 | 6% | 10 mg/mL | 26.40 ± 0.50 | 0.08 ± 0.01 |
| 4 | 6% | 10 mg/mL | 29.22 ± 0.43 | 0.09 ± 0.01 |

### Encapsulation Efficiency

The amount of propofol that could be loaded in the micelles was determined using HPLC. Propofol-loaded micelles were passed through membranes (2 times) to remove any unencapsulated propofol from the solution. The solution was then incubated in a tube with hydrophobic surface so that unencapsulated propofol sticks to the hydrophobic surface. HPLC was performed on the micellar solution, before and after the removal process to quantify propofol. The encapsulation efficiency for the propofol micelles was determined to be 96.2±1.3%.

### HPLC Optimization

An HPLC method for quantifying propofol was optimized and validated. The initial parameters optimized were mobile phase, flow rate and injection volume. A mobile phase of acetonitrile (70%), water (10%), and methanol (20%) was selected at a flow rate of 0.7 mL/min and injection volume of 20 μL. Propofol was quantified using UV detector by measuring absorbance at 270 nm.

Propofol Extraction from Serum Samples

A solvent containing 70% acetonitrile, 10% water, and 20% methanol was added to serum samples to crash out the proteins. The resulting solution was centrifuged at 6500 RPM for 20 mins. The supernatant was collected and passed through 0.22 μm filters (2 times) and loaded in HPLC vials for further analysis.

### Calibration Curve

A calibration curve of propofol added to serum samples was prepared. The calibration curve was linear between concentration range of 10 μg/mL to 250 μg/mL with an R2 value of 0.9993 (FIG. 4).

### Cytocompatibility

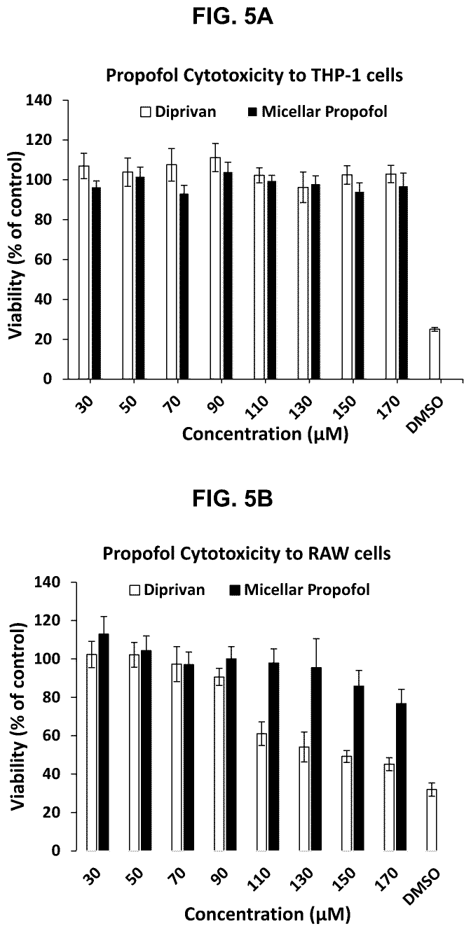

Micellar propofol formulation was tested for its in-vitro toxicity in human and murine macrophages and micellar propofol demonstrated similar cytocompatibility to Diprivan® in human macrophages at clinically relevant concentrations of propofol. Moreover, micellar propofol showed no toxicity to murine macrophages at higher concentrations whereas Diprivan® demonstrated toxicity to murine macrophages (FIG. 5A-B).

### ECMO Ex-Vivo

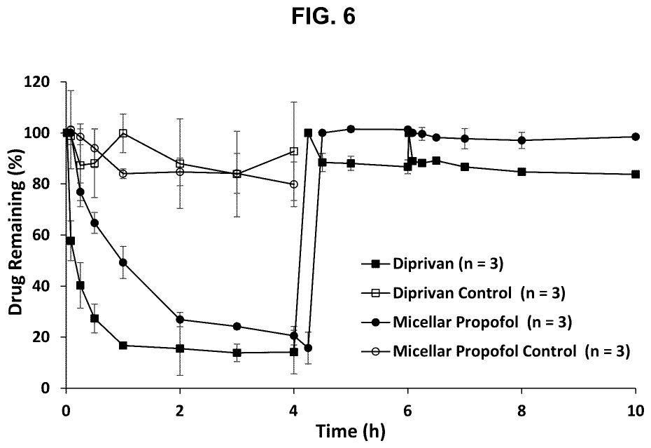

ECMO circuits were run with micellar propofol (n=3) or Diprivan® (n=3). The ECMO circuits were prepared as follows: human packed red blood cells, fresh frozen plasma, plasmalyte, heparin, sodium bicarbonate, tromethamine, calcium gluconate and albumin were combined in an ex-vivo ECMO circuit to mimic ECMO conditions. The ECMO circuit comprised of an oxygenator, pump, and tubing. The blood flow was maintained at 1 L/min and a temperature of 37° C. After priming the circuit, propofol micelles or Diprivan® were injected as a bolus dose to achieve therapeutic concentrations, and samples were collected at different time points. At 4 hours, an infusion dose of micellar propofol or Diprivan® was started which went on for 2 hours. After 2 hours the infusion was discontinued, and the circuit was allowed to run for another 4 hours, and samples were collected at different time points both during and after the infusion. Collected blood samples were centrifuged immediately a 3000×g, 4° C.) and plasma samples were stored at −80° C. until needed for further analysis. During analysis, propofol was extracted form plasma samples as mentioned. The samples were then analyzed for propofol concentration using HPLC per the above method, and then converted to propofol remaining (propofol recovery) using the equation below. The percentage of remaining propofol was then plotted against time (FIG. 6). The statistics of FIG. 6 are shown in Table 3.

$$Drug remaining ( \% ) = \frac{{C}_{t}}{{C}_{i}} \times 100 ,$$

where Cₜ is concentration at time T and Cᵢ is concentration at time T=1 min.

TABLE 3
Recovery of Propofol from Micellar Propofol and Diprivan

| Time (min) | P value | Significance |
|---|---|---|
| 1 | — | — |
| 5 | 0.0174 | * |
| 15 | 0.01 | ** |
| 30 | 0.01 | ** |
| 60 | 0.004 | ** |
| 120 | 0.227 | ns |
| 180 | 0.08 | ns |
| 240 | 0.8086 | ns |
| 255 | — | — |
| 270 | 0.0038 | ** |
| 300 | 0.0015 | ** |
| 360 | 0.001 | *** |
| 361 | — | — |
| 365 | 0.0025 | ** |
| 375 | 0.002 | *** |
| 390 | 0.02 | * |
| 420 | 0.006 | ** |
| 480 | 0.0001 | **** |
| 600 | 0.0033 | ** |

### Significance

The formulation significantly reduced the adsorption of propofol to the ECMO circuits compared to Diprivan®. For both formulations there was a rapid decrease in propofol recovery after a bolus dose with much higher recovery during and after the 2-hour continuous infusion. The results suggest that propofol adsorption is saturable and higher exposures can be achieved with a continuous infusion.

The difference in propofol adsorption between formulations is of unknown clinical significance. These experiments demonstrate that micellar encapsulation can decrease adsorption of drugs in the ECMO circuit. Encapsulation with a Generally Recognized As Safe (GRAS) micellar platform will allow rapid translation to clinical practice. This approach will transform drug development in critically ill patients on ECMO by minimizing drug adsorption by the circuit and optimizing dosing in this vulnerable population.

### Example 3

### Micelle Formation Using Other Pluronics and Propofol

Micelles were formed using propofol and Poloxamer P188. 600 mg of Poloxamer P188 (BASF, Germany) was added to 10 mL PBS (pH 7.4, VWR Life Science, USA) solution in a glass vial to obtain a 6% (w/v) formulation of P188 micelles. Propofol (2,6-diidopropylphenol, Sigma Aldrich, USA) was then added to the glass vials to obtain a desired concentration of 10 mg/mL of propofol. The mixture was placed under constant rotation overnight at 100 RPM in 4° C. using a magnetic stirrer (VWR, USA). The micellar solution was then filtered through 0.22 μm hydrophobic PTFE membrane filters (Membrane solutions, USA). Mean hydrodynamic diameter and polydispersity index (PDI) were determined by Dynamic Light Scattering (DLS) using a Malvern Zetasizer Nano-ZS (Malvern, Worcestershire, UK), as shown below in Table 4.

TABLE 4
Size and PDI of 6% P188 with 10 mg/mL Propofol

| P188 | Propofol | Z-Ave (d, nm) | PDI |
|---|---|---|---|
| 6% | 10 mg/ml | 32.3 ± 4.3 | 0.33 ± 0.02 |

### Example 4

### Micelles Formed Using a Combination of Poloxamer P407 and P188 at Different Ratios

A similar method (as described above) was used to formulate the micelles using a combination of P407 and P188 at different ratios. The ratios and their corresponding size and PDI are provided in Table 5 below. Further, these formulations were also tested for their colloidal stability for 7 days. For colloidal stability, the micellar solution in PBS was stored under constant shaking at 100 rpm at 37° C. for one week in an incubator shaker (Amerex Instruments, USA). Aliquots were collected directly after manufacturing and at each of the following 7 days, and subsequently characterized for their size and PDI via DLS.

TABLE 5
Stability of P407 and P188 Micellar Solutions in PBS

| | | | | After Day 7 | |
|---|---|---|---|---|---|
| P407 | P188 | At Day 0 | | (stability test) | |
| (%) | (%) | Size (nm) | PDI | Size (nm) | PDI |
| 100 | 0 | 25.9 ± 0.3 | 0.11 ± 0.02 | 26.1 ± 0.2 | 0.11 ± 0.01 |
| 90 | 10 | 26.4 ± 0.2 | 0.11 ± 0.01 | 27.00 ± 0.8 | 0.12 ± 0.01 |
| 80 | 20 | 30.9 ± 0.40 | 0.11 ± 0.00 | 32.1 ± 0.9 | 0.14 ± 0.01 |
| 70 | 30 | 34.8 ± 0.6 | 0.1 ± 0.01 | 35.9 ± 1.4 | 0.11 ± 0.01 |
| 60 | 40 | 41.3 ± 1.2 | 0.10 ± 0.01 | 40.8 ± 1.2 | 0.10 ± 0.01 |
| 50 | 50 | 42.9 ± 0.9 | 0.08 ± 0.37 | 42.4 ± 1.3 | 0.09 ± 0.01 |
| 40 | 60 | 44.2 ± 1.1 | 0.08 ± 0.01 | 42.8 ± 0.9 | 0.08 ± 0.01 |
| 30 | 70 | 48.2 ± 0.9 | 0.07 ± 0.01 | 40.4 ± 0.4 | 0.13 ± 0.01 |
| 20 | 80 | 54.4 ± 1.0 | 0.16 ± 0.01 | 42.9 ± 0.8 | 0.10 ± 0.01 |
| 10 | 90 | 31.3 ± 3.5 | 0.18 ± 0.02 | 33.5 ± 6.8 | 0.22 ± 0.04 |
| 0 | 100 | 32.3 ± 4.3 | 0.33 ± 0.02 | 31.8 ± 5.5 | 0.32 ± 0.04 |

The PDIs for each formulation do not change significantly from Day 0 to Day 7, suggesting that the formulations are stable at physiological temperatures (i.e., 37° C.) for at least 7 days.

The micelle size increases as the concentration of P188 increases, because P188 is a more hydrophilic polymer compared to P407. P407 is more suitable to encapsulate propofol because it is more hydrophobic.

### Example 5

Micelles with Varying Concentrations of Poloxamer P407

Previously, only micelles with 6% P407 were formulated. It was important to determine the highest concentration of P407 that can be used before the formulation starts gelling, because P407 is known to form gels above 20% (w/v) concentration at 37° C. A similar method as described above was used to formulate the micelles with varying concentrations of P407. Concentrations were prepared at 10%, 11%, 12%, 13%, 14%, 15%, 16%, 17%, 18%, and 19% P407. Tilt tests were performed to evaluate the gelation kinetics, if present, for all formulations.

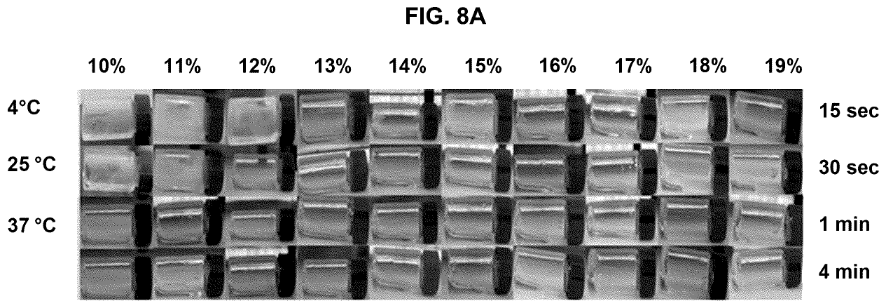

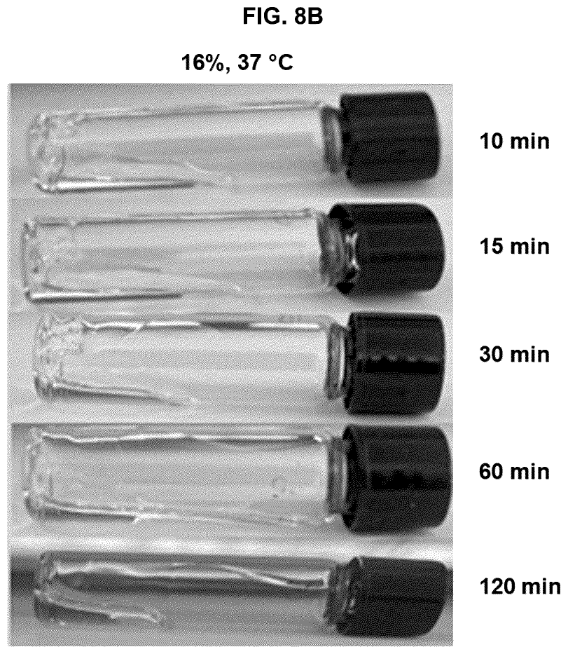

As observed in the tilt test (FIG. 8A), the formulations with P407 concentrations of 17%, 18%, and 19% P407 gelled immediately (in 1 min).

The 16% P407 formulation presented with questionable viscosity and was tested for higher time points for tilt test (FIG. 8B). It still was highly viscous after 120 minutes of tilt test and was not considered for a viable option for using as a formulation for ECMO circuits.

Formulations with 10%, 11%, 12%, 13%, 14%, and 15% did not gel at 37° C. However, the addition of propofol (10 mg/mL) to these formulations, resulted in changes in the gelation kinetics. The P407 15% and 16% (w/v) formulations gelled after addition of propofol, which is as expected because addition of small molecules to poloxamers are known to alter their physiochemical properties. The P407 14% sample did not gel with 10 mg/mL propofol, which means that 14% (w/v) may be the highest concentration that may be used to formulate micelles with 10 mg/mL of propofol. The size and PDI of these micelles are as follows:

TABLE 6
Size and PDI of 14% P407 with 10 mg/mL Propofol

| P407 | Propofol | Z-Ave (d, nm) | PDI |
|---|---|---|---|
| 14% | 10 mg/ml | 28.93 ± 1.24 | 0.04 ± 0.01 |

### Example 6

P407 and P188 Micelles with Other Drugs

Micelles with other drugs were formulated as described herein.

TABLE 7
P407 Micelles with Meropenem

| P407 | Meropenem | | Z-Ave (d, nm) | PDI |
|---|---|---|---|---|
| 6% | 5 | μg/mL | 28.8 ± 1.2 | 0.40 ± 0.06 |
| 6% | 25 | μg/mL | 27.3 ± 1.9 | 0.43 ± 0.06 |
| 6% | 50 | μg/mL | 29.5 ± 2.3 | 0.39 ± 0.11 |
| 6% | 1 | mg/mL | 27.2 | 0.34 |

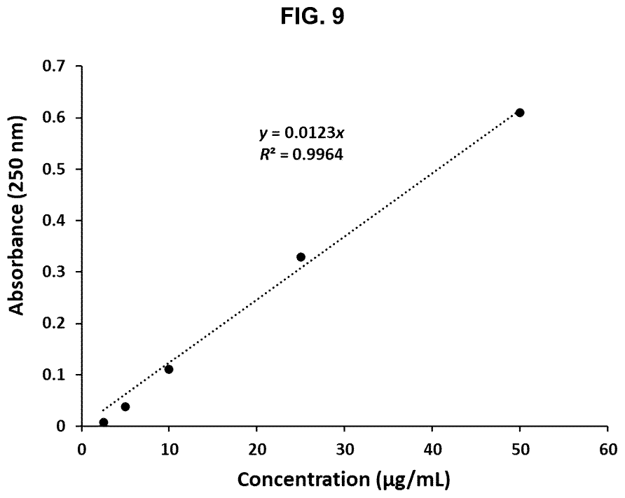

A calibration curve of meropenem was determined at 250 nm wavelength as shown in FIG. 9.

### Encapsulation Efficiency of Meropenem

Micellar meropenem (1 mg/mL) was dialyzed for 2 days using a float-a-lyzer (Repligen) to remove unencapsulated meropenem. Meropenem concentration was quantified pre- and post-dialysis using the calibration curve in FIG. 9. The encapsulation efficiency was determined to be 22.8%.

Meropenem when encapsulated in P407 micelles, did not have a good size distribution of micelles. Meropenem is not hydrophobic enough to efficiently encapsulate inside the hydrophobic core of P407 micelles.

### Example 7

### Liposomal Encapsulation of Meropenem

PEGylated liposomes (PL) were synthesized using the thin film hydration method. Briefly, 1,2-dipalmitoyl-sn-glycero-3-phosphocholine (DPPC), cholesterol and 1,2-distearoyl-snglycero-3-phosphoethanolamine-N-[methoxy(poly (ethylene glycol))-2000] (DSPE-PEG2000) (67:27:6 mass ratio) were dissolved in chloroform. Chloroform was evaporated to form a thin layer of the lipid mixture. Next, meropenem (5 mg/mL) and 10 mL of MilliQ water was added to the dry lipid film to obtain PEGylated liposomal meropenem. The flask was stored overnight for efficient hydration of the lipids. The following day, the lipid solution was subjected to sonication for 40 minutes using a water bath sonicator (Branson, Missouri, USA). Average size and polydispersity were measured by Dynamic Light Scattering (DLS) using a Malvern Zetasizer Nano-ZS (Malvern, Worcestershire, UK) by a standard protocol.

TABLE 8
Size and Polydispersity of Liposomal Meropenem

| | Downsizing | | |
|---|---|---|---|
| Formulation | Method | Z-Ave (d, nm) | PDI |
| Liposomal meropenem | Raw batch | 299 ± 25.7 | 0.8 ± 0.16 |
| Liposomal meropenem | After 40 min | 192.3 ± 15.2 | 0.52 ± 0.11 |
| | sonication | | |

Liposomal meropenem was lyophilized and mass of solid compound was measured. Following that, liposomal meropenem was solubilized in 3 mL of MilliQ water and dialyzed against a 500 Da membrane cut-off dialysis membrane (Repligen). The resultant solution was lyophilized, and the mass was measured.

$$Encapsulation efficiency = \frac{Mass of liposomes before dialysis - mass of liposome after dialysis}{total mass pf meropenem incorporated}$$

The encapsulation efficiency was determined to be 31.9%.

Based on these experiments, sonication helped in improving the PDI and size of the liposomes. Extrusion man need to be performed to further improve the size distribution of the liposomes. The encapsulation efficiency of meropenem in liposomes is 31.9%, which is be higher than meropenem in micelles. This was a very preliminary attempt to encapsulate meropenem in liposomes and further optimizations can be performed to increase the encapsulation efficiency

TABLE 9
Size and Polydispersity of P407 Micelles with Ampicillin

| P407 | Ampicillin | | Buffer | Z-Ave (d, nm) | PDI |
|---|---|---|---|---|---|
| 6% | 2 | mg/ml | PBS | 36.7 ± 3.8 | 0.48 ± 0.02 |
| 6% | 5 | mg/mL | PBS | 30.4 ± 2.6 | 0.48 ± 0.02 |
| 6% | 2 | mg/mL | Tris | 20.4 ± 0.8 | 0.13 ± 0.02 |
| 6% | 5 | mg/mL | Tris | 19.9 ± 0.07 | 0.14 ± .01 |

When PBS is used as buffer, the micelles are not monodispersed. When Tris is used as buffer, the micelles are highly monodispersed with PDI values below 0.2.

TABLE 10
Size and Polydispersity of P407
and P188 Micelles with Furosemide

| P407 | Furosemide | Z-Ave (d, nm) | PDI |
|---|---|---|---|
| 6% | 1 mg/ml | 19.5 ± 2.2 | 0.26 ± 0.01 |
| P188 | Furosemide | Z-Ave (d, nm) | PDI |
| 6% | 1 mg/ml | 6.4 ± 0.5 | 0.27 ± 0.03 |

Furosemide is a comparatively hydrophobic drug (log P=2.2) and hence can be encapsulated in micelles. Both P407 and P188 demonstrate similar PDI with encapsulation of furosemide, however, P188 shows a significantly smaller size of micelles after encapsulation.

### Example 8

### Liposomal Encapsulation of Propofol

PEGylated liposomes (PL) were synthesized using the thin film hydration method. Briefly, 1,2-dipalmitoyl-sn-glycero-3-phosphocholine (DPPC), cholesterol and 1,2-distearoyl-snglycero-3-phosphoethanolamine-N-[methoxy(poly (ethylene glycol))-2000] (DSPE-PEG2000) (67:27:6 mass ratio) were dissolved in chloroform. Chloroform was evaporated to form a thin layer of the lipid mixture. Next, propofol (100 μL) and 10 mL of MilliQ water was added to the dry lipid film to obtain PEGylated liposomal propofol. The flask was stored overnight for efficient hydration of the lipids. The following day, the lipid solution was subjected to sonication for 40 minutes using a water bath sonicator (Branson, Missouri, USA) followed by extrusion with membranes of 200 nm and 100 nm pore sizes (T&T scientific, SA). Average size and polydispersity were measured by Dynamic Light Scattering (DLS) using a Malvern Zetasizer Nano-ZS (Malvern, Worcestershire, UK) by a standard protocol.

TABLE 11
Size and Polydispersity of Liposomal Encapsulated Propofol

| Formulation | Downsizing Method | Z-Ave (d, nm) | PDI |
|---|---|---|---|
| Liposomal Propofol | Raw batch of liposomes | 382.8 ± 32.9 | 0.88 ± 0.1 |
| Liposomal Propofol | After 40 min sonication | 140.4 ± 4.8 | 0.22 ± 0.02 |
| Liposomal Propofol | Extrusion from 200 nm membrane | 141.1 ± 3.1 | 0.20 ± 0.01 |
| Liposomal Propofol | Extrusion from 100 nm membrane | 153.5 ± 5.5 | 0.18 ± 0.02 |

Raw batches of liposomes demonstrated a very high PDI, indicating high variability in the size of liposomes. After sonication for 40 minutes above the transition temperature of the lipid (41° C.), the PDI of the liposomes improved significantly and was close to the expected PDI of 0.2. Performing extrusion on the liposomes using a 200 nm and 100 nm pore size membrane did not change the size or improve the PDI of the liposomes. Hence, sonicating the raw batch is enough to obtain a fairly monodisperse formulation of liposomal propofol.

### Example 9

### PEGylation of Propofol

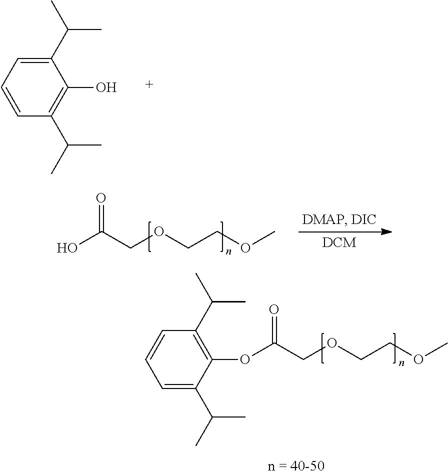

80 mg methoxy PEG acetic acid (MW 2000; n=40-50), 40 μL propofol, 4 mg of 4-dimethylaminopyridine (DMAP), and 40 μL of Diisopropylcarbodiimide (DIC) were dissolved in 5 mL of Dichloromethane (DCM). The reaction mixture was left for 24 hours under constant stirring. After 24 hours, the reaction mixture was washed 2 times with saturated Sodium Chloride (NaCl) solution using a separating funnel. Any remaining water was dried using anhydrous sodium sulphate. The organic layer was collected and was concentrated in rotary evaporator. Concentrated product was dissolved in 1 mL of methanol. Cold diethyl ether was added in excess to precipitate out the reaction product. The product was filtered and left to dry. Downstream characterizations were performed to confirm the completion of the reaction. Practical yields of reaction mixture after synthesis were >90%.

Infrared spectroscopy was performed using a Bruker Alpha attenuated total reflectance Fourier transformed infrared (ATR-FTIR). ¹H Nuclear magnetic resonance spectroscopy was performed using a Varian mercury spectrophotometer (400 Hz) and chemical shift were reported relative to CDCl₃. UV-vis spectroscopy was performed using a Spectramax M2 microplate reader (Molecular Devices).

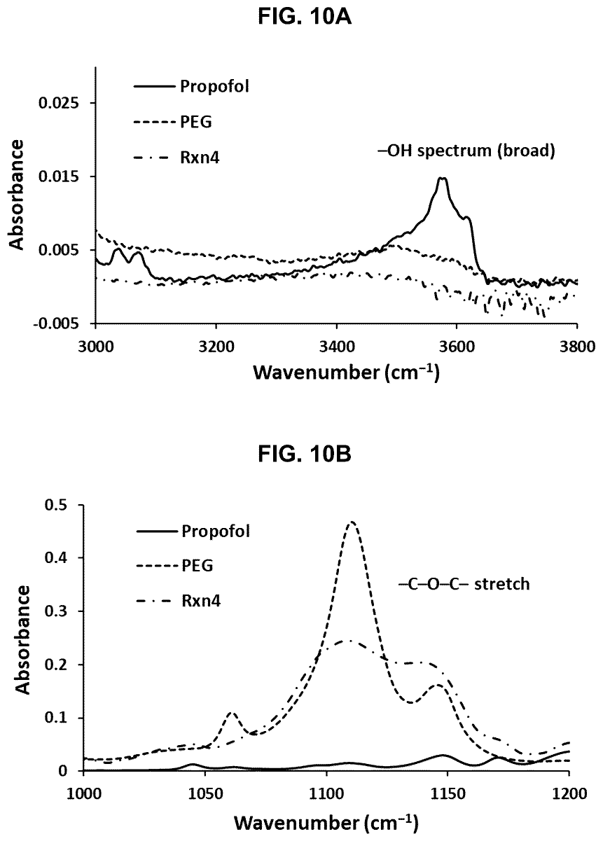

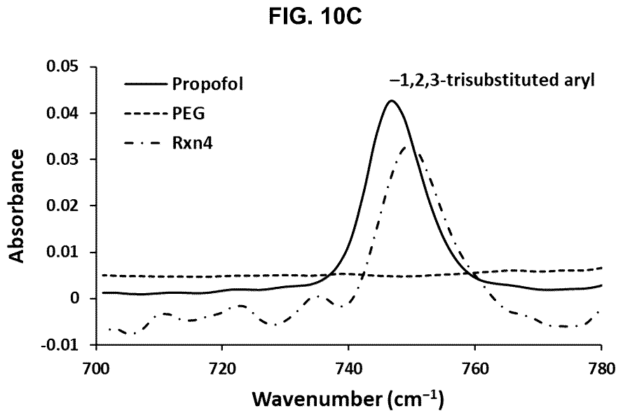

FTIR experiments of propofol shows a peak at 3600 cm⁻¹, which is a characteristic broad-spectrum peak for alcohol (—OH) group (FIG. 10A). However, Rxn4, which is the reaction product (PEG-propofol) does not show that peak, indicating that-OH is missing from the reaction product, hence indicating towards completion of PEGylation of propofol. The —C—O—C-stretch is maintained in both just PEG and PEG-propofol (FIG. 10B), which indicates both PEG and the reaction product demonstrates characteristics of —C—O—C-stretch, which appears because of

PEG. Finally, the peak at 780 cm-1 arises due to 1,2,3-trisubstituted aryl compounds, which in this case was from propofol (FIG. 10C). As can be observed in FIG. 10C, both propofol and the reaction product (PEG-propofol) show the 1,2,3-trisubstituted aryl compounds peak, while PEG does not. This confirmed the completion of PEGylation of propofol with no remaining free propofol.

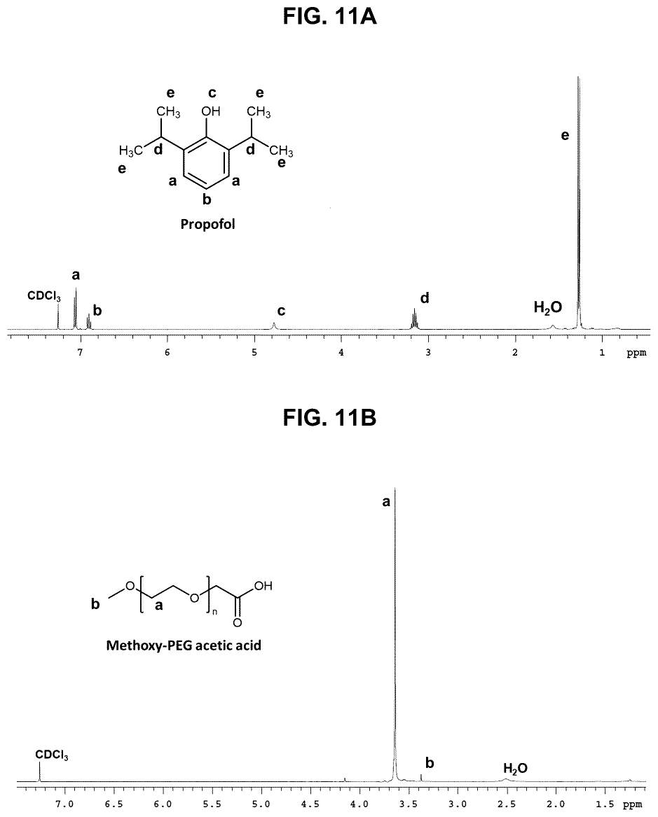

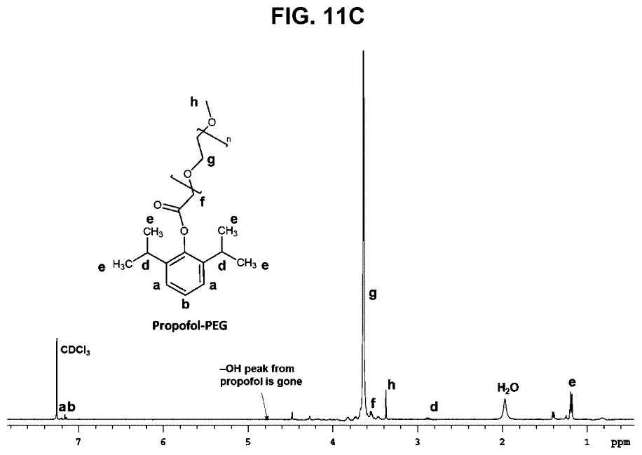

NMR experiments of propofol showed the characteristic chemical shift of an aryl-OH group at 4.8 ppm, whereas that peak is missing in the NMR spectrum of the reaction product, while the other chemical shift peaks of propofol such as the propyl groups, benzene hydrogens, and chemical shift peaks of PEG such as the methoxy hydrogen are maintained in the reaction product (FIG. 11A-C).

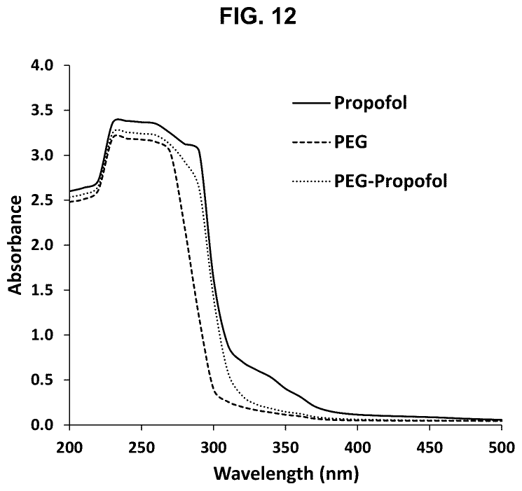

UV-visible spectroscopy showed a UV absorbance of PEG-propofol at exactly half value between propofol and PEG at 270 nm (FIG. 12). The shift in UV absorbance from PEG to PEG-propofol indicates PEGylation of propofol.

1. A pharmaceutical composition for administration in extracorporeal membrane oxygenation (ECMO) circuits, the composition comprising propofol encapsulated in an amphipathic encapsulating agent.

2. The pharmaceutical composition of claim 1, wherein the amphipathic encapsulating agent comprises a polymeric micelle or a liposome.

3. The pharmaceutical composition of claim 2, wherein the polymeric micelle comprises a polyethylene-polypropylene glycol micelle.

4. The pharmaceutical composition of claim 3, wherein the polyethylene-polypropylene glycol micelle is Poloxamer 407 (P407).

5. The pharmaceutical composition of claim 3, wherein the polyethylene-polypropylene glycol micelle is Poloxamer 188 (P188).

6. The pharmaceutical composition of claim 3, wherein the polyethylene-polypropylene glycol micelle is a mixture of P407 and P188.

7. The pharmaceutical composition of claim 2, wherein the liposome comprises a PEGylated liposome.

8. The pharmaceutical composition of claim 1, wherein the composition is stable for at least about 7 days at about 37° C.

9. The pharmaceutical composition of claim 1, wherein the composition is stable for at least about 6 months at about 2° C. to about 8° C.

10. The pharmaceutical composition of claim 1, wherein the composition has reduced adsorption in ECMO circuits as compared to compositions comprising propofol that is not encapsulated in an amphipathic encapsulating agent.

11. The pharmaceutical composition of claim 2, wherein the polymeric micelle encapsulating the propofol has a size of about 20 nm to about 40 nm.

12. The pharmaceutical composition of claim 11, wherein the polymeric micelle encapsulating the propofol has a size of about 25 nm to about 35 nm.

13. The pharmaceutical composition of claim 11, wherein the polymeric micelle encapsulating the propofol has a size of about 20 nm to about 30 nm.

14. The pharmaceutical composition of claim 11, wherein the polymeric micelle encapsulating the propofol has a size of about 30 nm to about 40 nm.

15. The pharmaceutical composition of claim 2, wherein the polymeric micelle encapsulating the propofol has a polydispersity index of about 0.05 to about 0.5.

16. The pharmaceutical composition of claim 15, wherein the polymeric micelle encapsulating the propofol has a polydispersity index of about 0.05 to about 0.2.

17. The pharmaceutical composition of claim 2, wherein the polymeric micelle encapsulating the propofol has a zeta potential of about-2.50 mV to about-5.00 mV.

18. The pharmaceutical composition of claim 2, wherein the polymeric micelle is present in an amount of about 5 wt % to about 14 wt %.

19. The pharmaceutical composition of claim 2, wherein the liposome encapsulating the propofol has a size of about 120 nm to about 400 nm.

20. The pharmaceutical composition of claim 2, wherein the liposome encapsulating the propofol has a polydispersity index of about 0.05 to about 0.5.

21. The pharmaceutical composition of claim 1, wherein the propofol is present in an amount of about 5 mg/mL to about 15 mg/mL.

22. The pharmaceutical composition of claim 21, wherein the propofol is present in an amount of about 10 mg/mL.

23-45. (canceled)
